(19) United States
(12) Patent Application Publication
(10) Pub. No.: US 2006/0099666 A1
(43) Pub. Date: May 11, 2006

(54) Analyzer for the simultaneous enzymatic detection of closely related analytes

(75) Inventors: Pierre Douville, Quebec (CA); Francois Rousseau, Quebec (CA); Jean-Claude Forrest, Quebec (CA)

Correspondence Address: FULBRIGHT & JAWORSKI, LLP, 600 CONGRESS AVENUE, SUITE 2400, AUSTIN, TX, 78701, US

(73) Assignee: Medinov, Inc., Quebec (CA)

(21) Appl. No.: 10/528,218

(22) PCT Filed: Sep. 19, 2003
(86) PCT No.: PCT/CA03/01429

§ 371 Date: Oct. 7, 2005

(30) Foreign Application Priority Data
Sep. 20, 2002 (US) 60411866

(51) Int. Cl. C12Q 1/26

(52) U.S. Cl. 435/25; 205/777.5

(57) ABSTRACT

An analyzer and a method for simultaneously detecting and measuring the concentration of two related analytes, the analytes being substrates for a common enzyme. The analyzer comprises an enzymatic reaction monitoring component including a support base, a mixed electrode system consisting of a working electrode, an auxiliary electrode and a reference electrode, the mixed electrode system being supported by said support base, and an enzymatic reaction means incorporating the enzyme, the enzymatic reaction means being disposed on the mixed electrode system; whereby, when the enzymatic reaction means is placed in contact with a liquid sample containing the two related analytes, the two related analytes chemically react with the enzyme to produce an electronic signal directly related to the concentration of each of the two related analytes in said liquid sample; a detector including a sensor, the detector being connected to the enzymatic reaction monitoring component and capable of continuously detecting and amplifying said electronic signal to produce amplified signals; and a data processor capable of converting the amplified signals into numerical data representative of the concentration of each of the two related analytes.

### FIELD OF THE INVENTION

The present invention relates to an analyzer for the simultaneous enzymatic detection of closely related analytes. More specifically, the present invention relates to an analyzer for the simultaneous enzymatic detection of methanol and ethanol.

### BACKGROUND OF THE INVENTION

Methanol and ethanol are natural fermentation products. Methanol is produced from the distillation of wood, whereas ethanol is produced from the fermentation of sugars. All alcoholic beverages containing ethanol are still made by this process. Both ethanol and methanol are clear and colorless liquids. Methanol is a constituent of many commercially available solvents such as windshield wiper fluids and deicers, antifreeze, glass cleaner, as well as paints and paint thinners. Its concentration may be up to 300 mg per liter in wine and may even be higher in other spirits. Ethanol is commonly used in the manufacture of some car fuels, perfumes and paints.

Methanol and ethanol are readily absorbed from the gastro-intestinal tract as well as through the skin. Alcohol dehydrogenase metabolizes methanol to the toxic metabolites formaldehyde and formic acid, whereas ethanol is metabolized to acetaldehyde. Formic acid is responsible for the profound metabolic acidosis that is typical of methanol poisoning. The overall mortality of methanol poisoning is approximately 20% and, among survivors, the rate of permanent visual impairment is about 20 to 25%.

The simultaneous assessment of ethanol and methanol represents an important detection and measurement challenge frequently encountered by medical institutions having to respond to intoxication related problems. The ability to simultaneously assess both alcohol concentrations in biological samples would considerably improve the medical intervention in point-of-care centers facing situations where methanol intoxication is suspected.

The clinical symptoms of methanol intoxication, within the first one to two hours, may be similar to ethanol intoxication. Therefore, the diagnosis of methanol ingestion is rendered difficult since the symptoms and physical signs are non-specific. Early visual disturbances, including decreased or blurred vision, are the classic findings that are associated with methanol intoxication.

Most clinical laboratories possess the equipment for quantitatively measuring the ethanol concentration in blood or urine samples. However, methanol is measured only in reference centers using sophisticated instrumentation such as for example a gas chromatograph. Most emergency rooms are susceptible to require methanol measurements in order to rule out any possible methanol intoxication. Consequently, it has become imperative that these point of care centers possess the ability to rapidly make such an assessment.

Early recognition of methanol poisoning, that is as soon as possible after ingestion, is essential since any substantial delays can often become detrimental and have to be avoided for appropriate intervention. Specific antidote treatment exists and has to be undertaken without delay. Even though this condition is rare, an investigation for methanol intoxication is frequently carried out in emergency rooms on alcoholics, drug addicts, as well as on various other types of patients displaying unspecific neurological symptoms.

Park et al. (U.S. Pat. No. 5,571,395) describe a breath analyzer comprising a biosensor for measuring alcohol concentrations in exhaled gas, having selectivity for ethanol. The breath analyzer uses electrochemical principles capable of measuring the drinking degree, by electrochemically reacting with the alcohol contained in the exhaled gas.

Hayashi et al. (U.S. Pat. No. 5,081,015) disclose an enzyme electrode as well as a method for determining the alcohol (ethanol) content in a sample. The enzyme electrode is described as having an immobilized enzyme layer including a crosslinked reaction product of an alcohol oxidase (alcohol oxidase solution), a crosslinking agent, and reduced glutathione. The enzyme electrode determines the alcohol content present in the sample by measuring the amount of hydrogen peroxide produced by the oxidation of the alcohol by the alcohol oxidase.

McAleer et al. (U.S. Pat. No. 6,241,862 and U.S. Pat. No. 5,951,836) teach a disposable glucose test strip for use in a test meter performing electrochemical determinations of blood analytes such as glucose.

Yamauchi et al. (U.S. Pat. No. 5,609,749) teach an electrochemical assay method (enzyme electrode) for measuring a substance such as for example glucose or cholesterol, in a liquid biological sample. The assay method essentially comprises an oxidation-reduction enzyme, an electron mediator (p-phenylenediamine), and an electrode. p-Phenylenediamine compounds are disclosed as being used as the electron mediators due to their inherently large electron transfer rates with enzymes.

Nankai et al. (U.S. Pat. No. 5,185,256 and U.S. Pat. No. 4,897,173) disclose a disposable biosensor for the quantitative determination of specific components in biological samples. More precisely, the biosensor can be applied to systems associated with oxidoreductases such as for example glucose oxidase and alcohol oxidase.

Winarta et al. (WO 00/73785) disclose a disposable electrode strip including an enzyme and an electron mediator. The strip further includes an interference-correcting electrode, minimizing any interference caused by additional oxidizable species present in the sample fluid. The electrode's response is substantially independent of the hematocrit levels of the sample.

Pritchard et al. (WO 97/02487) disclose an electrochemical biosensor strip that can be used for determining glucose levels. The biosensor strip includes a working and a counter electrode having essentially the same size, and which are made of the same electrically conducting material. The strip additionally includes a reagent well, exposing a smaller area of the counter electrode than of the working electrode. The strip has the advantage of a lower minimum blood requirement than prior art strips of similar construction.

None of these prior art references teaches a device or a method capable of simultaneously detecting closely related analytes such as methanol and ethanol.

There thus remains a need to develop a device for the simultaneous enzymatic detection of closely related analytes such as methanol and ethanol. Furthermore, there remains a need to develop a method capable of differentially detecting closely related analytes such as methanol and ethanol, quickly and accurately.

The present invention seeks to meet these and other needs.

The present invention refers to a number of documents, the content of which is herein incorporated by reference in their entirety.

### SUMMARY OF THE INVENTION

The present invention relates to an analyzer for simultaneously detecting and measuring the concentration of two related analytes, the analytes being substrates for a common enzyme, comprising:

(a) an enzymatic reaction monitoring component including a support base, a mixed electrode system consisting of a working electrode, an auxiliary electrode and a reference electrode, the mixed electrode system being supported by said support base, and an enzymatic reaction means incorporating the enzyme, the enzymatic reaction means being disposed on the mixed electrode system; whereby, when the enzymatic reaction means is placed in contact with a liquid sample containing the two related analytes, the two related analytes chemically react with the enzyme to produce an electronic signal directly related to the concentration of each of the two related analytes in said liquid sample;

(b) a detector including a sensor, the detector being connected to the enzymatic reaction monitoring component and capable of continuously detecting and amplifying said electronic signal to produce amplified signals; and

(c) a data processor capable of converting the amplified signals into numerical data representative of the concentration of each of the two related analytes.

The present invention relates to a method for simultaneously detecting and measuring the concentration of at least two related analytes in a sample, the related analytes being substrates for a common enzyme, wherein the enzyme reacts with the related analytes following specific different reaction kinetics, and wherein the method comprises:

(a) reacting a plurality of reference samples having known concentrations and proportions of the related analytes, the proportions ranging from 0 to 100% of a first analyte to 100% to 0% of another related analyte, with said enzyme;

(b) establishing a kinetic profile having at least two points for each of said plurality of reference samples; and

(c) reacting a test sample comprising an unknown concentration and proportion of the related analytes with the enzyme and determining the concentration of the related compounds in the test sample using the established kinetic profiles.

The present invention relates to an enzymatic reaction monitoring component for simultaneously detecting and measuring the concentration of two related analytes, the analytes being substrates for a common enzyme, comprising:

(a) a support base;

(b) a mixed electrode system consisting of a working electrode, an auxiliary electrode and a reference electrode, the mixed electrode system being supported by the support base; and

(c) an enzymatic reaction means incorporating the enzyme, the enzymatic reaction means being disposed on the mixed electrode system; whereby, when the enzymatic reaction means is placed in contact with a liquid sample containing the two related analytes, the two related analytes chemically react with the enzyme to produce an electronic signal directly related to the concentration of each of the two related analytes in the liquid sample.

In one embodiment, the present invention relates to a portable analyzer for the simultaneous enzymatic detection and measurement of methanol and ethanol in biological samples.

In a second embodiment, the present invention relates to a non-portable analyzer for the simultaneous enzymatic detection and measurement of methanol and ethanol in biological samples.

The present invention relates to an analyzer capable of detecting and measuring the presence of related analytes such as for example methanol and ethanol, for use in point-of-care units, in laboratories, in police services, in forensic applications and in industrial applications.

The present invention also relates to a method for the simultaneous detection and measurement of closely related analytes.

In a third embodiment, the present invention relates to a method for the simultaneous detection and measurement of methanol and ethanol in biological samples.

The present invention also relates to a method for the detection of closely related analytes, more specifically methanol and ethanol, by differential enzymatic measurements thereof.

In a fourth embodiment, the present invention relates to a method by which closely related analytes are detected and measured by the analysis of the chemical reaction dynamics, through multiple time points response measurements.

The present invention further relates to an electrode for the detection and simultaneous enzymatic measurement of closely related analytes.

In a fifth embodiment of the present invention, the present invention relates to an electrode for the detection and simultaneous enzymatic measurement of methanol and ethanol.

In addition, the present invention relates to a method for the simultaneous detection and measurement of two or more related analytes in a sample serving as a substrate to a common enzyme, wherein the enzyme reacts with the related analytes following specific reaction kinetics. The method comprises the steps of: (a) reacting a plurality of reference samples including known proportions of the two related analytes, ranging from 100% of a first analyte to 100% of a second analyte, with the enzyme; (b) establishing kinetic profiles having at least two points for each of the plurality of reference samples; (c) reacting a test sample which may include the related analytes with the enzyme and; (d) determining the concentration of each of the related analytes in the test sample using the previously established kinetic profiles.

In a sixth embodiment, the present invention relates to a method for the simultaneous detection and measurement of at least two related analytes in a sample, wherein the related analytes serve as substrates to a common enzyme, and wherein the enzyme reacts with the related analytes following different specific reaction kinetics. The method comprises the steps of: (a) reacting a plurality of reference samples including known concentrations and proportions of the related analytes, the proportions ranging from 0 to 100% of a first analyte to 100% to 0% of second related analyte, with the enzyme; (b) establishing a kinetic profile having at least two points for each of the plurality of reference samples; (c) reacting a test sample having an unknown concentration and proportion of the related analytes with the enzyme and; (d) determining the concentration of each of the related analytes in the test sample using the previously established kinetic profiles.

In addition, the present invention relates to an enzymatic reaction monitoring component (disposable or permanent) for simultaneously detecting and measuring at least two related analytes in a sample, the analytes being substrates to a common enzyme, the enzymatic reaction monitoring component comprising: (a) a support base and; (b) a mixed electrode system consisting of a working electrode, an auxiliary electrode and a reference electrode, wherein the working electrode and the auxiliary electrode are composed of platinum, wherein the reference electrode is composed of silver, and, wherein the mixed electrode system is being supported by the support base. The disposable enzymatic reaction monitoring component further includes a layer of a permeable polymer on which is bound an enzyme layer, deposited on the electrode system, as well as a protective membrane impregnated with a buffer and with reagents, disposed over the permeable polymer layer. In a further embodiment, the present invention relates to an enzymatic reaction monitoring component as defined hereinabove, for measuring methanol and ethanol.

In a sixth embodiment, the present invention relates to an enzymatic reaction monitoring component (disposable or permanent) for simultaneously detecting and measuring at least two closely related analytes in a sample, the analytes being substrates to a common enzyme, comprising: (a) a support base; (b) a mixed electrode system consisting of a working electrode, an auxiliary electrode and a reference electrode, wherein the working electrode and the auxiliary electrode are composed of platinum and wherein the reference electrode is composed of silver, and wherein the mixed electrode system is being supported by the support base and; (c) an enzymatic reaction means including the enzyme, the enzymatic reaction means further receiving reagents suitable to support a reaction between the analytes and the enzyme, and being capable of generating species detectable by the mixed electrode system, the means being in connection with the electrode system.

Furthermore, the present invention relates to an analyzer for the simultaneous enzymatic detection and measurement of closely related analytes, including a disposable or a permanent enzymatic reaction monitoring component comprising: (a) a support base and; (b) a mixed electrode system consisting of a working electrode, an auxiliary electrode and a reference electrode, wherein the working electrode and the auxiliary electrode are composed of platinum, wherein the reference electrode is composed of silver, and, wherein the mixed electrode system is being supported by the support base. The disposable enzymatic reaction monitoring component further includes a layer of a permeable polymer on which is bound an enzyme layer, deposited on the electrode system, as well as a protective membrane impregnated with a buffer and with reagents, disposed over the permeable polymer layer.

Further scope and applicability will become apparent from the detailed description given hereinafter. It should be understood however, that this detailed description, while indicating preferred embodiments of the invention, is given by way of illustration only, since various changes and modifications will become apparent to those skilled in the art.

### BRIEF DESCRIPTION OF THE DRAWINGS

Having thus generally described the invention, reference will now be made to the accompanying drawings, showing by way of illustration a preferred embodiment thereof, and in which:

FIG. 1 is a schematic illustration of an enzyme mediated redox reaction;

FIG. 2 is an illustration of a mixed electrode.

FIG. 3a is a schematic illustration of an electronic circuit for the detection and amplification of the electric signal generated by the electrode in the course of the enzymatic reaction; FIG. 3b is a schematic illustration of the main components of an electrode (strip or permanent) including electronic components, to be used with either a strip electrode or a permanent electrode;

FIG. 4 is a schematic illustration of various components of a disposable electrode in accordance with the present invention;

FIG. 5 is a schematic illustration of an analyzer in accordance with an embodiment of the present invention;

FIG. 6a is an illustration of a permanent mixed electrode system comprising a reference electrode made of silver and an auxiliary electrode and a working electrode made of platinum; FIG. 6b is an illustration of an analyzer integrating the electrode cell, the mixer arm, as well as the various electronic components for the amplification, computation of the concentrations, and lecture of the signal produced by the electrode;

FIG. 7 is a graph depicting the determination in triplicate of various methanol concentrations as a function of observed voltage over time. The figure further illustrates very good reproducibility of the observed results;

FIG. 8 is a graph depicting the determination of various methanol concentrations using a voltage amplification circuit specifically developed for the analyzer of the present invention;

FIG. 9 is a graph illustrating the improvement of the electronic signal, as obtained using a peroxymetric circuit, for a sample having a methanol concentration of 10 mM, by using potassium ferrocyanide (upper graphs) in conjunction with the enzyme;

FIG. 10 illustrates a Hanes diagram of the K_{M} for methanol, obtained electrochemically with Pichia pastoris;

FIG. 11 is a graph illustrating the reaction of various samples having different concentrations of methanol with 10 U/mL of Pichia pastoris;

FIG. 12 is a graph illustrating the reaction of various samples having different concentrations of ethanol with 10 U/mL of Pichia pastoris;

FIG. 13 is a graph illustrating the reaction of a various samples including respectively 0.025 mM of methanol and 1; 0.75; 0.5; 0.25; 0.1 mM of ethanol with 10 U/mL of Pichia pastoris;

FIG. 14 is a graph illustrating the reaction of various samples including respectively 0.05 mM of methanol and 1; 0.75; 0.5; 0.25; 0.1 mM of ethanol with 10 U/mL of Pichia pastoris;

FIG. 15 is a graph illustrating the reaction of various samples including respectively 0.075 mM of methanol and 1; 0.75; 0.5; 0.25; 0.1 mM of ethanol with 10 U/mL of Pichia pastoris;

FIG. 16 is a graph illustrating the reaction of various samples including respectively 0.1 mM of methanol and 1; 0.75; 0.5; 0.25; 0.1 mM of ethanol with 10 U/mL of Pichia pastoris;

FIG. 17 is a graph illustrating raw voltage measurements using a Pt/Ag electrode and samples having various alcohol concentrations, versus time;

FIG. 18 is a graph illustrating an alcohol dosage and proportion analysis plot, for different alcohol solutions;

FIG. 19 is a graph illustrating various response curves for methanol solutions having concentrations ranging from 1-7 mM, expressed as voltage measurements versus time;

FIG. 20 is a graph illustrating various response curves for ethanol solutions having concentrations ranging from 545 mM, expressed as voltage measurements versus time;

FIG. 21 is a graph illustrating differential kinetic responses for methanol and ethanol solutions, expressed as voltage measurements versus time;

FIG. 22a is a diagram illustrating the response (expressed in volts) as a function of ethanol concentration, measured at fixed time intervals; FIG. 22b is a diagram illustrating the response (expressed in volts) as a function of methanol concentration, measured at fixed time intervals; and

FIG. 23 is a diagram illustrating standard curves for ethanol and methanol.

FIG. 24 is a diagram illustrating standard curves for ethanol and methanol, and including an example of the signal from a sample containing a mixture of both alcohols.

Other objects, advantages and features of the present invention will become more apparent upon reading of the following non-restrictive description of preferred embodiments, with reference to the accompanying drawings, which is exemplary and should not be interpreted as limiting the scope of the present invention.

### DESCRIPTION OF THE SPECIFIC EMBODIMENT

The terms “electron transfer agent” and “electron transfer mediator” are used interchangeably herein.

The present invention relates to an analyzer as well as to a method for simultaneously detecting and measuring closely related analytes such as ethanol and methanol. The quantification and detection of the related analytes is carried out through continuous kinetic measurements, as well as through the continuous analysis of a signal from a single enzymatic reaction, since both analytes serve as substrates to a common enzyme. The method and the analyzer, due to their accuracy, sensitivity, as well as their rapidity to operate and provide results, are useful for applications in point-of-care units, in laboratories, in police services, in forensic applications as well as in industrial applications.

The present invention has been exemplified by its capacity to differentiate alcohols, namely ethanol and methanol. In no way should this invention be limited to alcohol detection as the basic principles of this invention can be applied to other enzymes and other substrates, common to each of these enzymes.

The specificity of enzymes coupled to electronic detection devices can considerably improve the detection limit for certain molecules, for example hydrogen peroxide. Enzyme mediated reactions generate molecules capable of being oxidized at the surface of an electrode, resulting in the formation of a measurable electronic current. This current can subsequently be amplified, analyzed and interpreted by a specialized microprocessor.

The analyzer of the present invention essentially comprises a disposable or permanent electrode which, for instance, is an alcohol biosensor including miniaturized electrodes and reagents, a signal amplification system and a data analyzer.

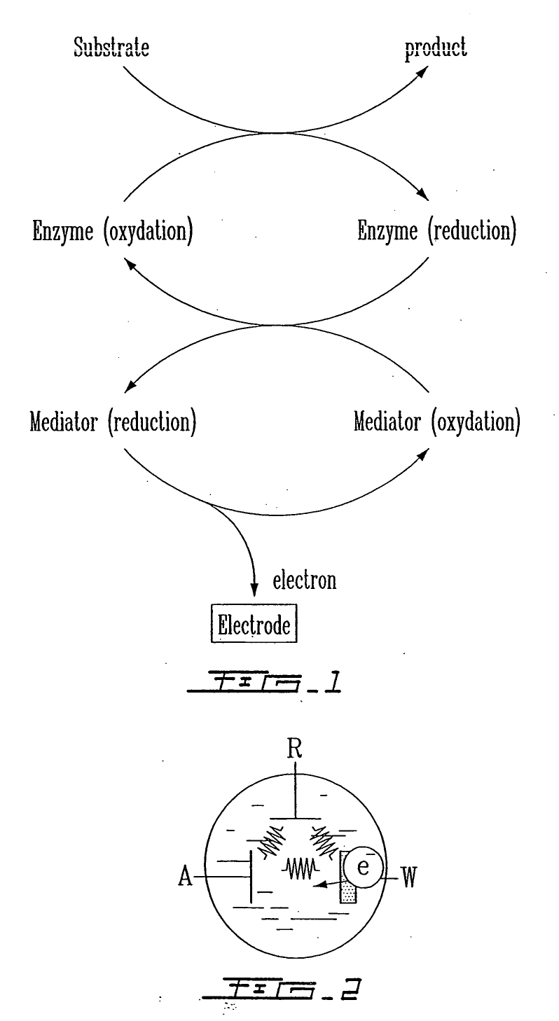

Alcohol oxidase, which can be optionally incorporated into a miniaturized disposable electrode, reacts with methanol and ethanol following specific reaction kinetics, producing a signal that is continuously registered and converted into data points by a detector. A schematic illustration of an oxidase catalyzed redox reaction is provided in FIG. 1. Oxidase mediated reactions are particularly adaptable to the production of biosensors since these enzymes catalyze redox reactions with electron transfer.

A biosensor is essentially a device comprising a compound of biological origin (for example an enzyme, an antibody, a protein or a nucleic acid) and an electronic detector. The compound of biological origin is in intimate contact with the detector, and provides for a chemical reaction upon binding to a substrate or ligand as illustrated below in the case of an alcohol (Scheme 1). The enzymatic reaction results in the formation of a metabolite (hydrogen peroxide) which in turn provides for an electronic signal that is subsequently captured by the detector.

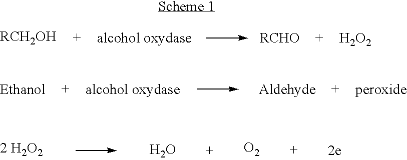

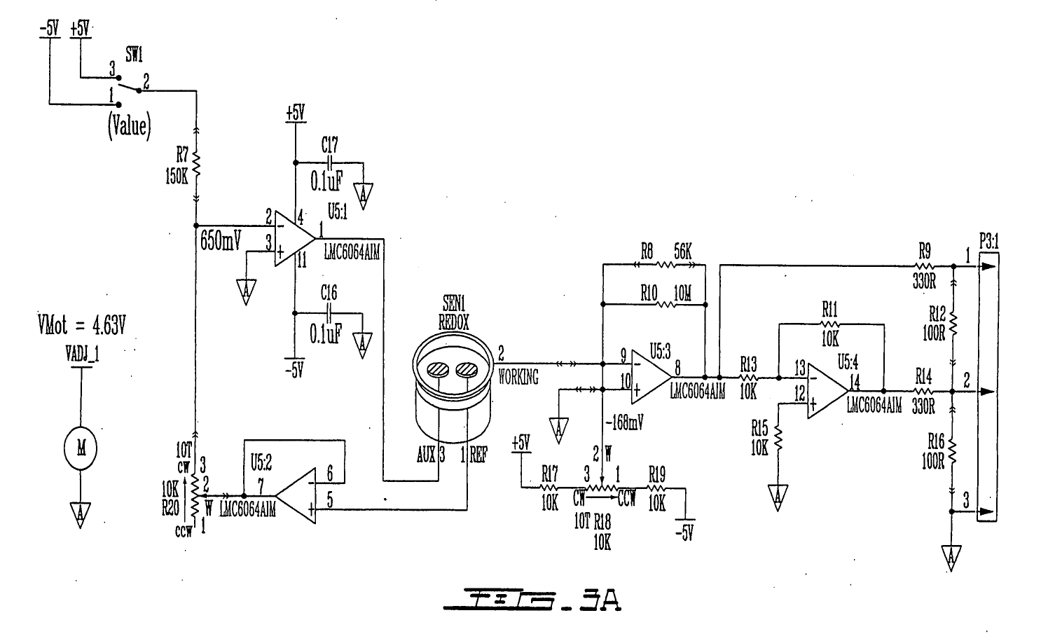

The electrode in accordance with the present invention, can either be a single-use device (disposable strip) or a multiple-use device, and comprises a system of three electrodes, of which one, the reference electrode, serves to control the signal to noise ratio. Good results are obtained by a system of three electrodes wherein one is composed of silver (reference electrode) and the remaining two are composed of platinum (the working electrode and the auxiliary electrode). Such a system of electrodes is known as a “mixed electrode”, and is depicted in FIG. 2.

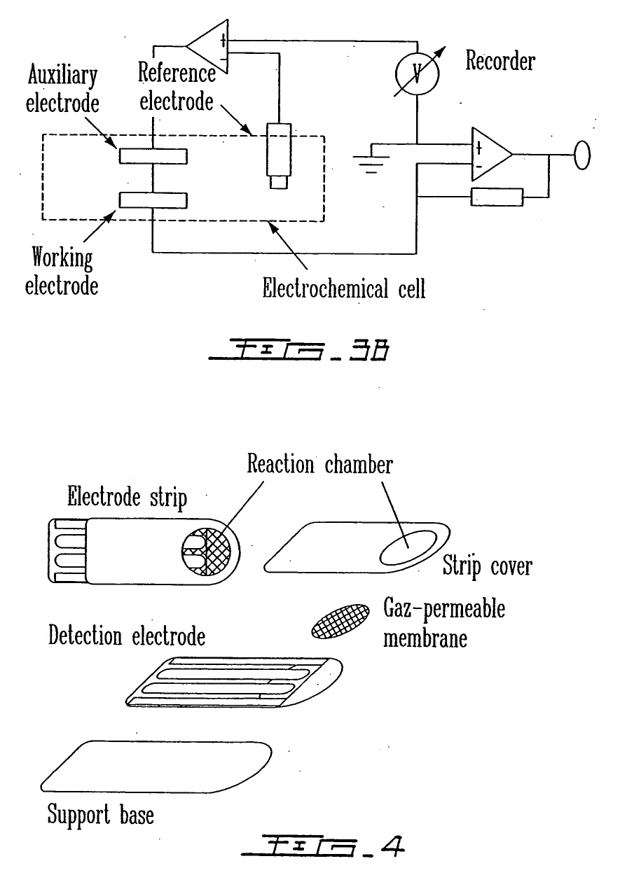

The redox reaction takes place on the working electrode (W). This electrode either receives or donates electrons, that is compounds either become reduced or become oxidized at this electrode. The auxiliary electrode (A) corrects variations in the reaction potential caused by the reaction medium, whereas the reference electrode (R) maintains a constant oxidation potential at the working electrode. An electrochemical detector comprising a mixed electrode is illustrated in FIGS. 3a and 3b. Platinum (working electrode and the auxiliary electrode) is the metal that best responds to peroxide, produced by the enzymatic reaction of alcohol oxidase. Silver, on the other hand, is the standard metal for the reference electrode. The auxiliary electrode is made of an identical metal as the working electrode, since it has to provide a comparable response.

A particularly important characteristic of the analyzer of the present invention, is its capacity to simultaneously measure two related substances, at occurrence methanol and ethanol, during a single enzymatic reaction. A signal is continuously measured during this reaction, followed by its mathematical conversion into concentration data for the two alcohols. The simultaneous determination is based on the principle that alcohol oxidase reacts with these alcohols following specific reaction kinetics. A careful analysis of the reaction of alcohol oxidase with ethanol and methanol, allows for the establishment of the precise reaction conditions required for the simultaneous determination of the concentration of both alcohols.

The analyzer of the present invention, comprising an enzyme disposed on a miniaturized electrode, and wherein the reaction of the enzyme with either or both alcohols is continuously monitored, constitutes a considerable improvement over the current art.

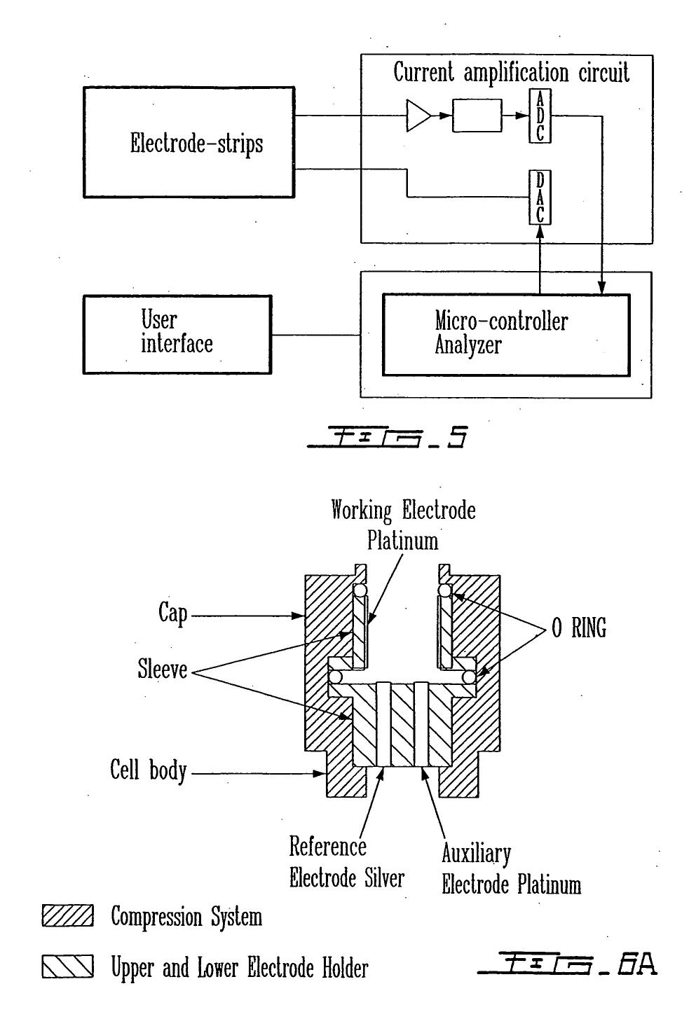

The development of specific electrodes, such as illustrated in FIG. 4 (disposable strip), for the simultaneous measurement of related alcohols, represents a substantial advancement in the art. Some advantages provided by the present system are its simplicity of operation, its specificity (i.e. capable of distinguishing among related analytes), its portability and its miniaturization.

The disposable electrode as illustrated in FIG. 4, comprises a mixed electrode that is deposited on a support base that can be made, for example, of plastic. In one particular embodiment, the electrode design consists of a detection electrode having no reagent incorporated, and which serves to monitor the peroxide concentration in a reaction chamber, in which a sample to be analyzed is added, along with the enzyme in buffer solution.

The electrode can additionally include an electron mediator, more specifically ferrocene or potassium ferrocyanide, for electron transfer towards the electrode, where a constant potential of approximately 200 to 300 mvolts will be usually generated. The electron mediator is usually incorporated in the reagent solution to be added to the sample on the electrode. Other reagents that could serve as electron mediators are selected from the group consisting of p-phenylenediamine, peroxidase and other ferrocene derivatives such as ferrocene dicarboxylic acid, and ferrocene monocarboxylic acid. Possible buffer solutions are selected from the group consisting of phosphates, saline phosphate buffers (phosphates+NaCl), TRIS-HCl, Hepes, with or without EDTA, or a wetting agent such as SDS, Triton X-100 or Tween 20.

A disposable electrode strip is inserted into a specifically adapted socket of the portable analyzer. A reagent solution, optionally comprising the enzyme is then added onto the electrode followed by the application of a biological specimen such as for example saliva, blood, or serum (as is, or diluted). The enzymatic reaction that is triggered by the application of the biological sample is monitored by a sensor, which will send sequential readings at a rhythm of hundreds per second to a digital processing device.

Alternatively, the enzyme and the reagents can be covalently attached or deposited (trapped/embedded) on the electrode, and the strip used directly with the biological sample without the need to add a reagent solution. For measurements in biological samples, the electrode chamber can be isolated from the sample cells and proteins by a permeable membrane or polymers. Possible permeable polymers that can function in this capacity are selected from the group consisting of polylysine, poly(4-styrene sulfonate), polyethylene glycol, perfluorosulfonic acid polymers and agarose.

The signal processing device or “DSP” includes a programmable electronic chip, capable of integrating mathematical algorithms essential for the conversion of the readings into numeric values of concentration of ethanol and methanol respectively.

In one particular embodiment, the analyzer of the present invention comprises a disposable or a permanent electrode (enzymatic reaction monitoring component), a detector, and a data processor for the conversion of the signal (readings) into numerical data (concentration of the respective analytes). The detector includes an amplifier that is coupled to a sensor containing a programmable chip. The detector is capable of detecting the electronic signal, generated by redox reaction taking place on the permanent or the disposable electrode, in a continuous or kinetic mode. The sensitivity of the portable analyzer renders it practical for the detection of minute amounts of a substance or condition to be identified. This system is further illustrated in FIG. 5.

Development of the Enzymatic Reaction Monitoring Component

In order to satisfy the requirements of the measurements to be carried out by the analyzer of the present invention, an enzymatic reaction monitoring component comprising a system of three electrodes is desirable.

In order to ensure and maintain a stable oxidation potential corresponding to the molecule to be oxidized at the working electrode, a reference electrode composed of silver is preferentially used. Since hydrogen peroxide (H₂O₂) is the molecule to be oxidized, an oxidation potential of about 650 mvolts is optimally maintained at the working electrode if no electron transfer agent is to be used. In the presence of an electron transfer agent, a lower oxidation voltage can be used, such as for example with ferrocyanide which only requires a potential of about 285 mvolts. This oxidation potential is specific to hydrogen peroxide, and ensures a maximal response from the electrode while minimizing any potential interference resulting from the oxidation of other compounds.

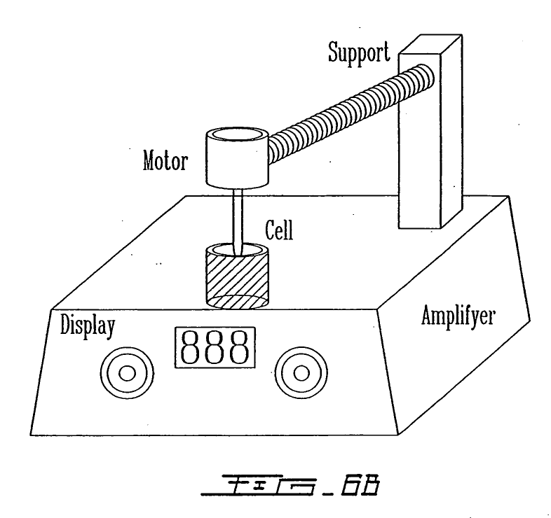

A mixed electrode system including a working electrode and an auxiliary electrode made of platinum, as well as a reference electrode made of silver, was developed and is illustrated in FIG. 6a. This selection of metals allows for the maintenance of an oxidation potential of about 650 mvolts, or of about 285 mvolts if an electron transfer agent is used. Furthermore, platinum is the metal that best responds to the peroxide produced by the alcohol oxidase reaction. The present system is both metal and mediator specific. More specifically, with platinum and silver, and in the absence of a mediator, a potential of about 650 mvolts is desirable. This electrode system is corrosion and abrasion resistant, and provides for a constant oxidation potential.

The size of the electrodes can be modified, depending on a particular need. Since the enzymatic reaction takes place throughout the entire reaction chamber when the enzyme is not fixed to the surface of the electrode, and the oxidation of hydrogen peroxide takes place only at the surface of the working electrode, it would be advantageous to maximize the surface area of the working electrode. This would allow for the measurement of increasingly smaller concentrations of alcohol. In the case where the enzyme is fixed to the surface of the working electrode, diffusion of the reaction products is less of a problem since they are already dose to the working electrode.

Methanol Determination Using the Newly Developed Electrode System

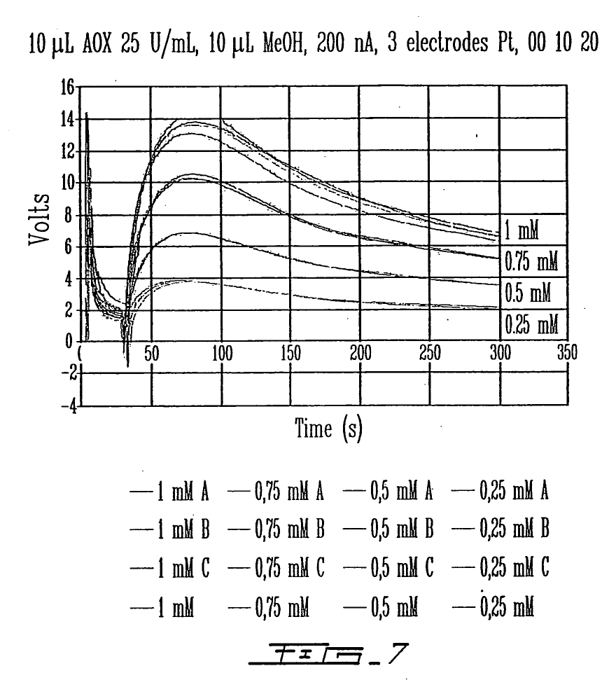

The results obtained for the determination of various concentrations of methanol in a series of experimental samples, using the newly developed electrode system, are illustrated in FIG. 7. As can be observed from FIG. 7, the detection limit for methanol was lowered to 0.25 mM. The reproducibility of the results was also very good, as shown by trials “A”, “B” and “C”, which all produced experimental curves in close proximity and shape to one another. Subsequent improvements to the electrode system, wherein the electrodes are directly manufactured from the corresponding metallic wires, allowed for a further lowering of the detection limit. Considering that the observed signal is dependent on the amplification by the electrochemical detector, at 200 nA, a methanol concentration of 0.01 mM and an ethanol concentration of 0.25 mM can be distinctly detected.

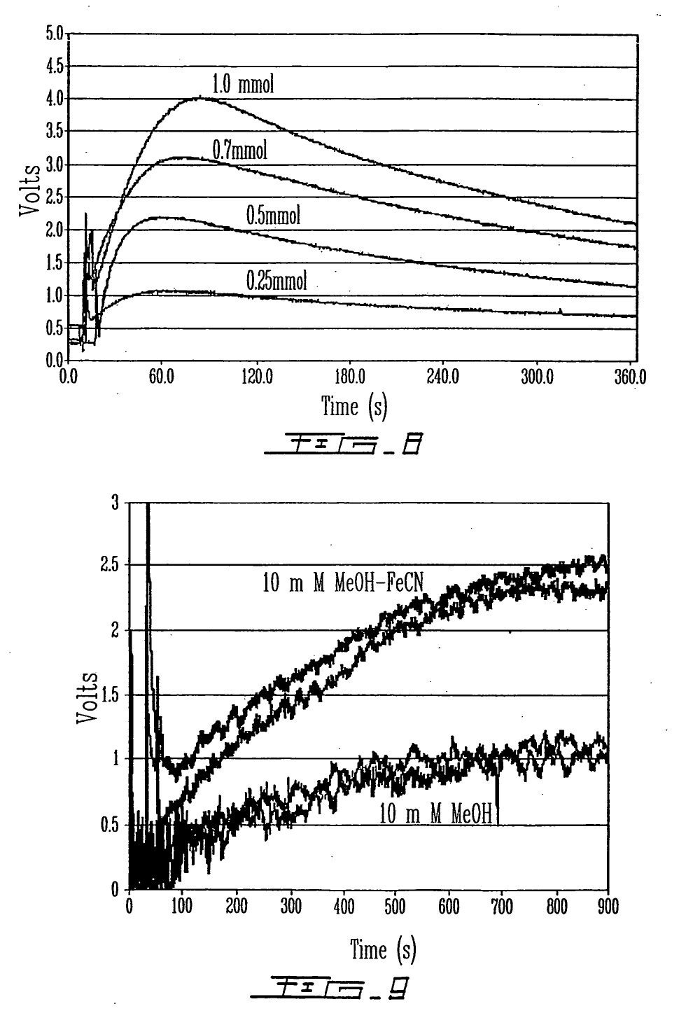

An electronic amplification circuit was specifically designed to amplify the electronic signal obtained from the alcohol oxidase catalyzed redox reaction. More precisely, a voltage amplification circuit was developed. The results obtained for the determination of various methanol concentrations using this circuit are illustrated in FIG. 8.

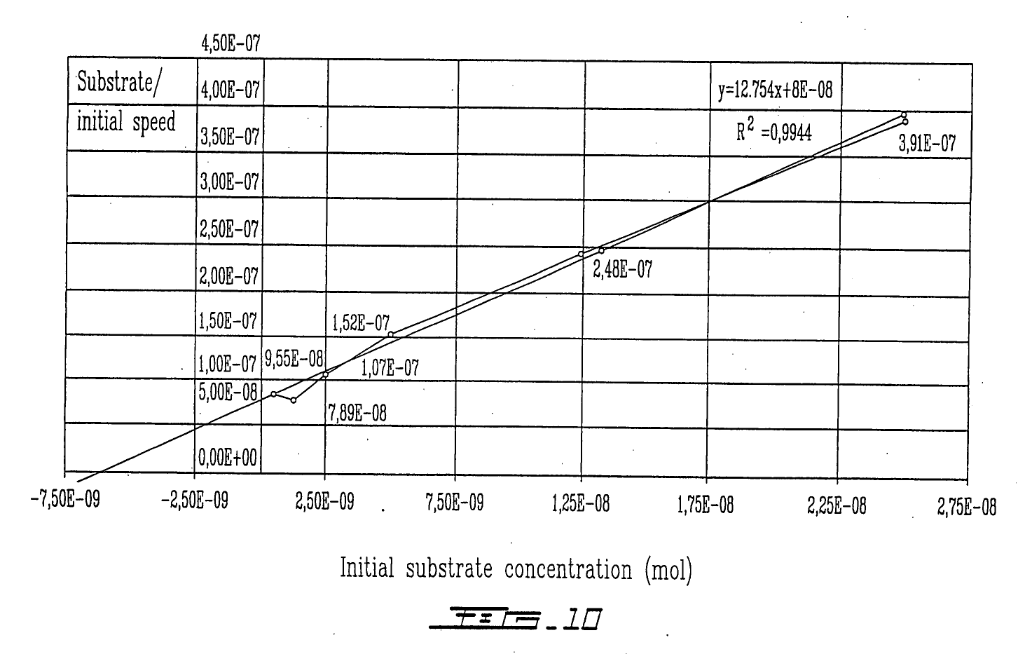

The electronic signal resulting form the oxidase catalyzed redox reaction can be further improved by incorporating an electron transfer reagent in the reaction medium. The intensity of the electronic signal can be increased by a factor of about 2.5 if potassium ferrocyanide is used in collaboration with the oxidase (FIG. 9). In the case where the electrode has no fixed enzyme, a buffered enzyme solution (10 μL of PBS/saline phosphate buffer, pH 7.4) was placed on the enzymatic reaction monitoring component. After waiting for about 30 seconds, a sample was added (10 μL) and the voltage measured in real time for up to 5 minutes. In the case where the electrode has an embedded enzyme on its surface, water (10 μL) was added followed by the addition of a sample (10 μL). The reaction was again monitored in real time. In a particular embodiment, the enzyme is embedded in an agarose matrix by applying a buffered enzyme solution (10 μL of PBS/saline phosphate buffer, pH 7.4) followed by drying.

Kinetic Studies

The Michaelis-Menten constant (K_{M}) of an enzyme can be determined in an aqueous environment from the initial reaction rate of the enzyme catalyzed reaction, by either calorimetric or electrochemical analysis. The initial reaction rate was determined using both methods, based on the results obtained for different concentrations of methanol and ethanol and various alcohol oxydases (Hansenula sp., Pichia pastoris and Candida boidinii). Using the Hanes method (diagram of the concentration/initial rate as a function of the initial concentration of the substrate in the reaction medium), the K_{M} can be calculated, which corresponds to the origin of the straight line, opposite the abscissa. The various experimental K_{M} values obtained using the Hanes method for various ethanol and methanol concentrations are shown in Table 1. A Hanes diagram of the K_{M} for methanol, obtained electrochemically with Pichia pastoris is illustrated in FIG. 10.

TABLE 1
Experimental K_{M} values obtained by the Hanes method.

| | K_{M} MeOH | | K_{M} EtOH | | |
|---|---|---|---|---|---|
| Enzyme | mM | [] mM | mM | [] mM | Method |
| Candida boidini | 0.29 | 0.05-0.5 | 8.41 | 0.25-1.25 | E¹ |
| Hansenula sp. | 0.39 | 0.05-2.5 | 6.00 | 0.25-5.0 | E |
| Pichia pastoris | 0.31 | 0.05-2.5 | 2.61 | 0.25-5.0 | E |
| Pichia pastoris | 0.56 | 0.125-1.0 | 2.91 | 2.5-10.0 | E |
| Pichia pastoris | 0.64 | 0.02-2.5 | 3.46 | 0.05-3.3 | C² |

¹Electrochemistry is abbreviated by “E”.
²Colorimetry is abbreviated by “C”.

Measurements were carried out on numerous samples of varying concentrations of methanol and ethanol, allowing for a comparison of the signals. It becomes obvious from the shape of the graphs, and from the knowledge that alcohols having longer carbon chains react slower, that methanol reacts faster than ethanol with an identical concentration of enzyme. This particularity, for an identical amplification factor, allows for the determination of various amounts of an alcohol with the help of a mathematical algorithm taking into account the intensity and dynamics of the signal generated by the enzymatic reaction.

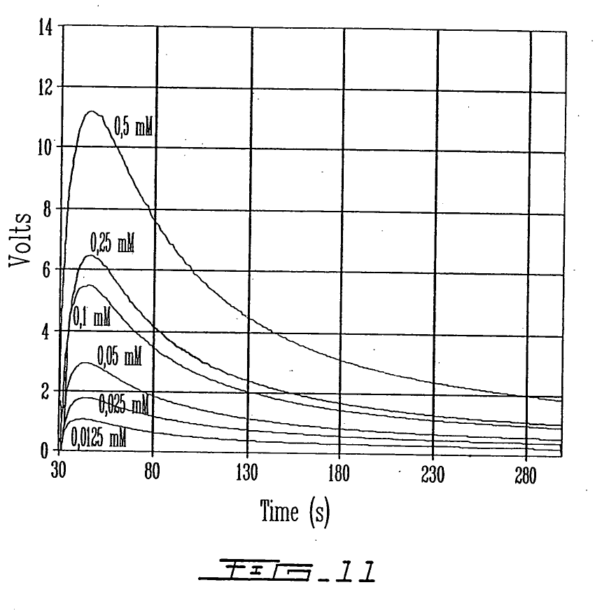

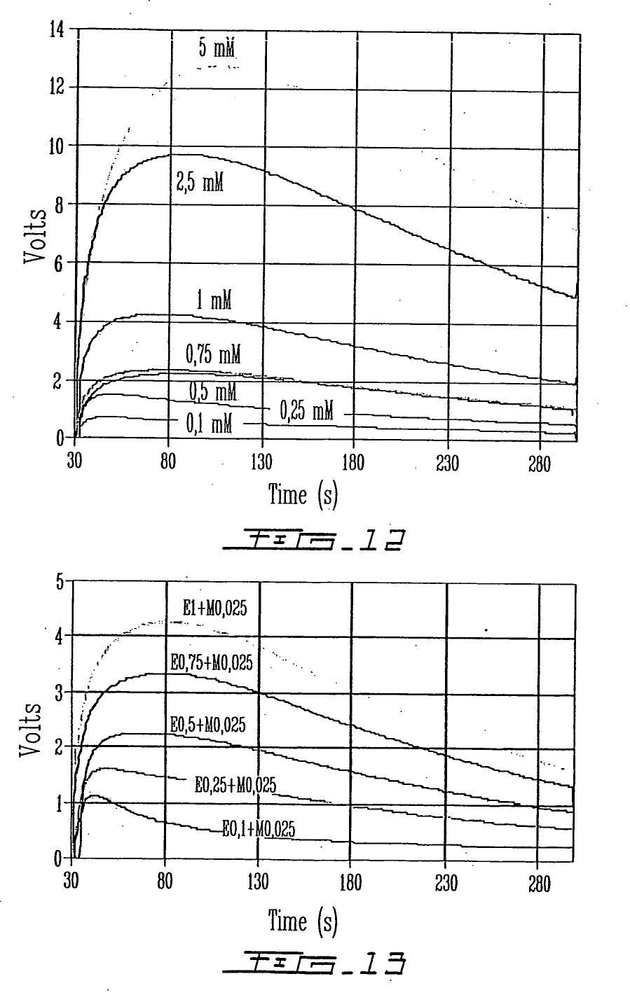

As can be observed from a comparison of the graphs shown in FIGS. 11 and 12, depicting the signal from the enzymatic reaction with various concentrations of methanol and ethanol respectively, methanol is consumed at a faster rate. A sharper peak is indicative of a faster reaction rate.

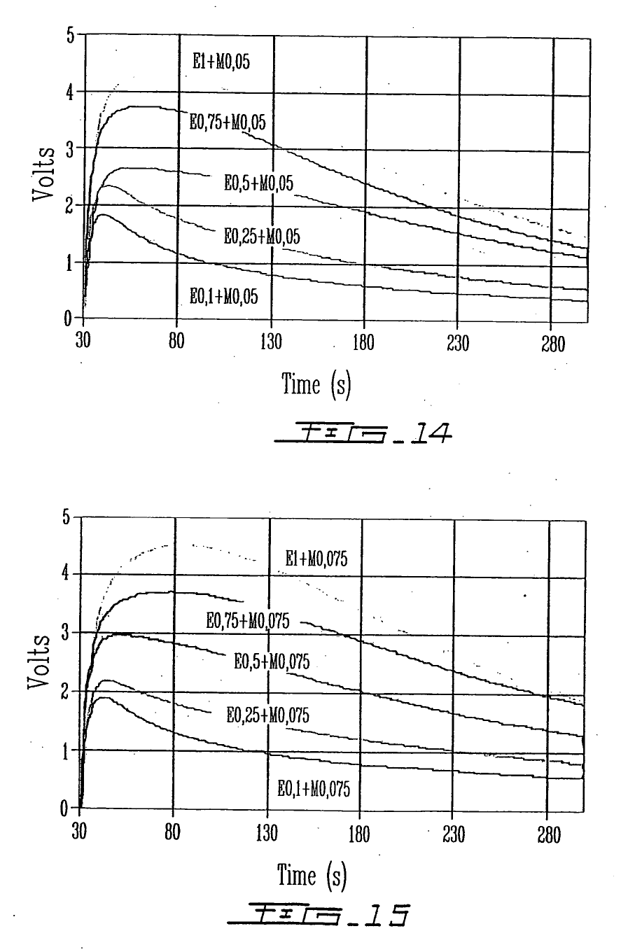

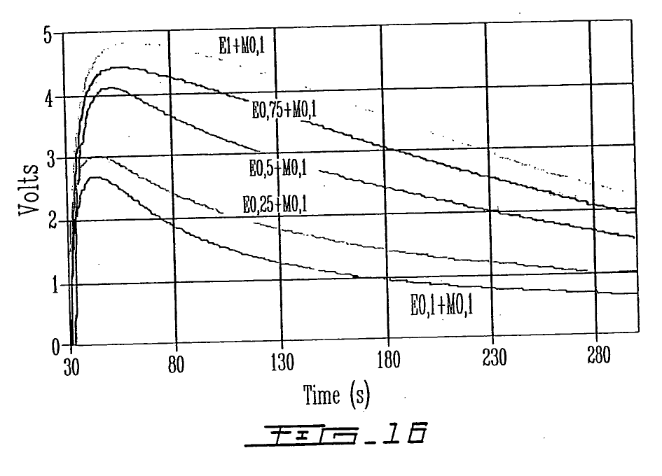

FIGS. 13 and 14 illustrate the effect of reducing the ethanol concentration on the shape of the graphs obtained for samples further comprising a methanol concentration of 0.025 mM and 0.05 mM respectively. It becomes readily apparent, that as the concentration of ethanol is reduced (1.0; 0.75; 0.5; 0.25; 0.1 mM) with respect to the methanol concentration, the shape of the graph increasingly resembles the graph obtained for methanol.

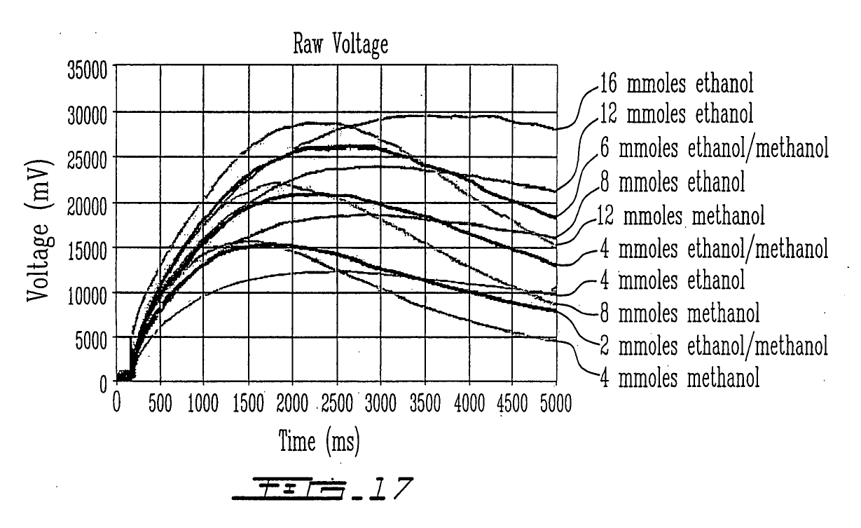

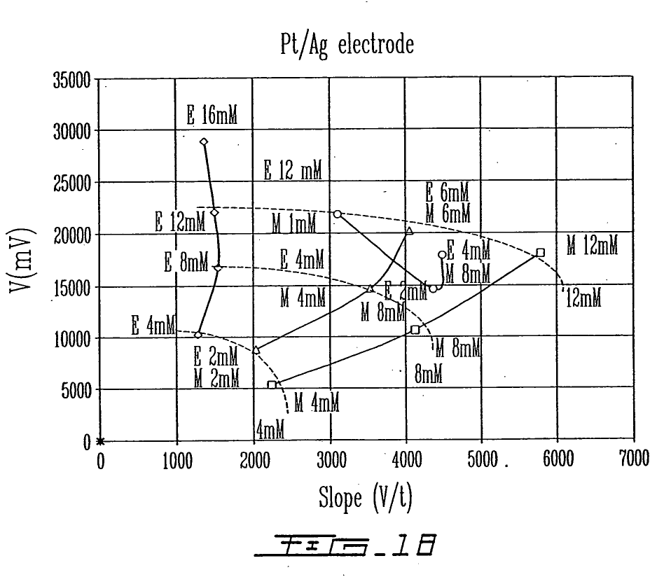

FIGS. 15 and 16 illustrate the effect of reducing the ethanol concentration on the shape of the graphs obtained for samples further comprising a methanol concentration of 0.075 mM and 0.1 mM respectively. It is again readily apparent in both cases, that as the concentration of ethanol is reduced (1.0; 0.75; 0.5; 0.25; 0.1 mM) with respect to the methanol concentration, the shape of the graph increasingly resembles the graph obtained for methanol. The sensitivity of the above described method is more than adequate for clinical use, since the concentration ranges to be detected, are clearly higher.

Development of a Mathematical Model

It is desirable to transform the electric current generated by the electrode into concentrations of methanol and ethanol. This can be achieved in various ways based on at least 2 readings at different times.

I) Comparison with a Set of Experimental Data

The unknown concentrations of methanol and ethanol in a sample can be determined by the comparison of the observed response with a set of experimental responses, obtained from solutions containing known concentrations of ethanol and methanol. The experimental set is such that it covers the concentration range of the two alcohols and mixtures thereof, in proportions ranging from 0 to 100%. The method consists of matching the response of the unknown sample with a member of the experimental set. The respective concentration of ethanol and methanol in the unknown sample is then derived from the experimental value of the experimental set that best fits the observed response.

II) Multiple Regression Analysis

It is possible to use a statistical tool such as regression analysis wherein ethanol and methanol are dependent variables, and wherein at least two response data points are used as independent variables. Regular statistical multiple regression analysis can be used to calculate regression coefficients from experimental results obtained with a set of solutions of ethanol and methanol of known concentration. The equation thus obtained can be used to calculate the concentration of unknown solutions. The exact parameters for this equation are recalculated at each calibration. Such a statistical method was developed and has produced an equation useful in determining the ethanol and methanol concentration in unknown samples, and is shown below:
Methanol=0.0309+(0.02529×Vₘₐₓ)−(0.00171×tₘₐₓ)
Ethanol=−0.299+(0.167×Vₘₐₓ)−(0.011×tₘₐₓ)
wherein Vₘₐₓ is the maximum voltage in volts (V), and tₘₐₓ is the time at maximum voltage in seconds (sec).
   III) Enzyme Kinetics Model

A mathematical model describing the reaction occurring at the working electrode during the co-deposition of the oxidase with an aliphatic short chain alcohol, was developed. The model was elaborated based on a series of equations based on the laws of classical enzymes kinetics.

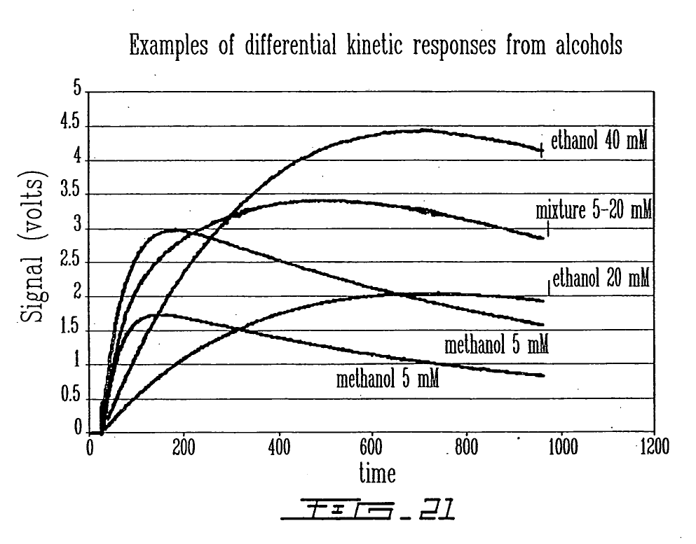

The model, in combination with the graph illustrating the current as a function of time (FIG. 21), allows for the determination of a curve depicting hydrogen peroxide formation as a function of substrate (methanol or ethanol). The observed current as a function of time was approximated using the following equations:
St=Eo[(Ke+Y)/Y−(Ke+Y)/Ye^{-KeT}−(1−e^{-YT})]
wherein:
Eo=initial concentration of ethanol;
Ke=activity constant for ethanol;
Y=constant related to the elimination of peroxide;
T=time (seconds); and
St=observed current (volts) at time=T
   St=Mo[(Km+Y)/Y−(Km+Y)/Ye^{-KmT}−(1−e^{-YT})]
   wherein:
Mo=initial concentration of methanol;
Km=activity constant for methanol;
Y=constant related to the elimination of peroxide;
T=time (seconds); and
St=observed current (volts) at time=T

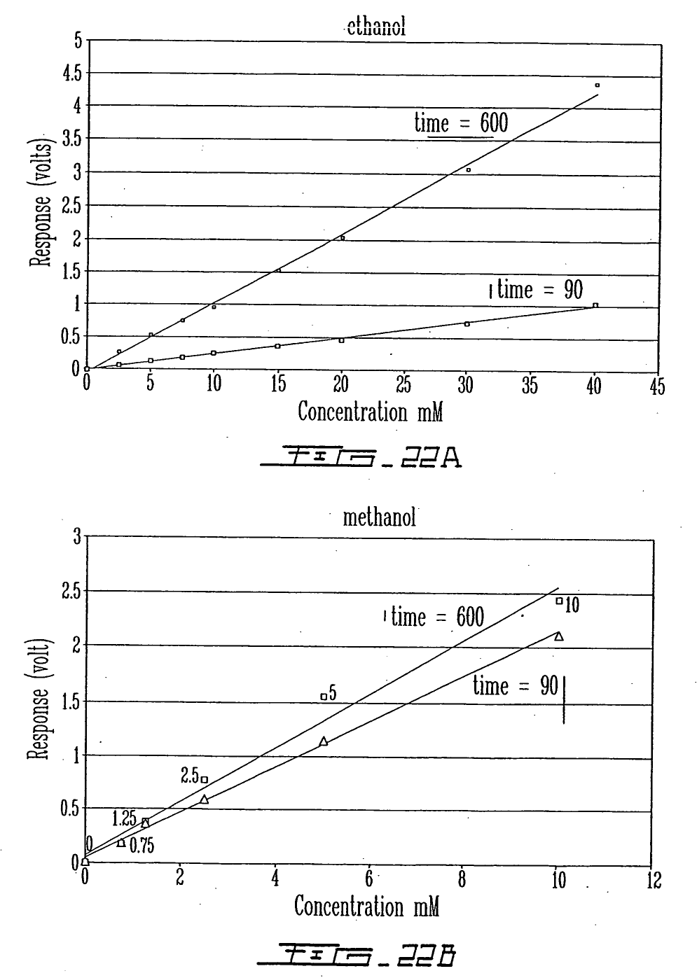

In the present system, the activity constant for methanol (Km) is lower than the activity constant for ethanol Ke. As can be seen observed in FIGS. 22a and 22b, for each time T, the observed signal (volts) is proportional to the amount of initial substrate (concentration). Both signals are additive, at least up to concentrations of about 40 mM for ethanol, and 10 mM for methanol. The difference in reactivity between the substrates serves as the basis for the determination of their respective amounts. In the simplest case, two time points can be used to calculate the respective quantity of each substrate.

The two time points are selected such as to represent early and late points. Preferentially, these time points are selected before and after the peak time of the most reactive substrate. It was found advantageous to use a first time point that represents about 57% of the peak time of the most reactive substrate, but other time points can also be selected. The choice of the second point is less critical, and can be before or after the peak time of the least reactive substrate.

Since the differential determination of the respective substrates is based on the reaction kinetics, it is not necessary to run the enzymatic reaction to completion. It can be advantageous to quickly select the second point, such that a rapid response is obtained for the user. The second point is preferably selected from the group of time points ranging from about 1 to about 10 times the peak time of the most reactive substrate, and most preferably from about 2 to about 6 times the peak time of the most reactive substrate.

Each measurement of a calibrator or an unknown sample generates a characteristic signal curve. The observed signal at the first and second time points (S1 and S2) can be used to generate a 2-dimensional calibrator plot. In the simplest case, the signals from both substrates are linear and additive, providing the following equations:
S1=AEo+BMo
S2=CEo+DMo
wherein A, C and B, D are calibration constants for ethanol and methanol respectively, established experimentally with pure solutions.

The values for Eo and Mo for unknown samples can be readily derived algebraically as follows:
Eo=(BS2−DS1)/(CB−DA)
Mo=(AS2−CS1)/(AD−CB)

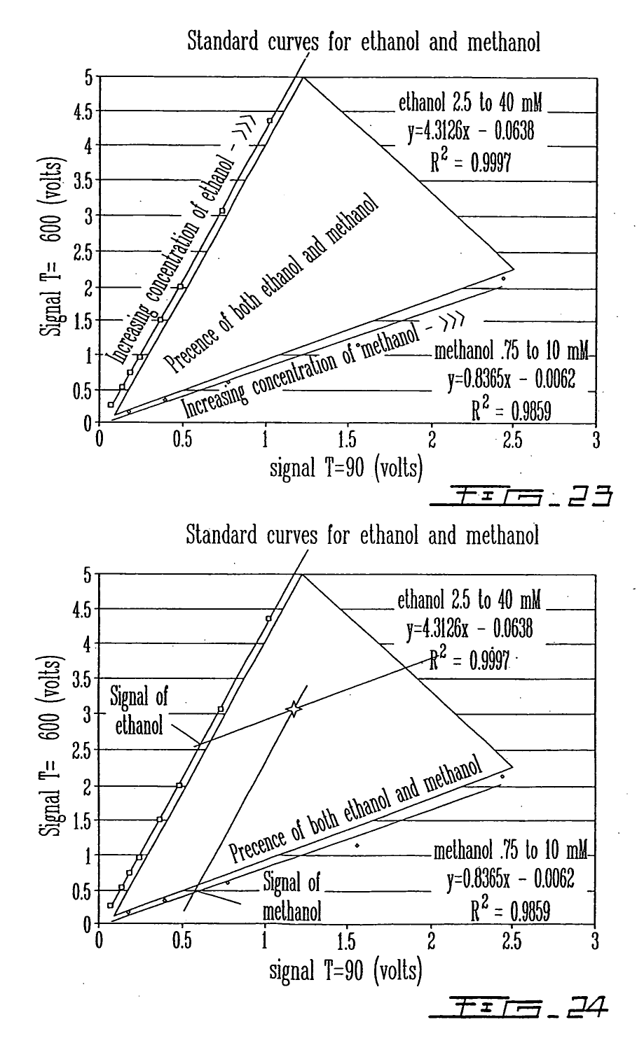

Furthermore, the signal ratio S2/S1 yields a constant value for pure solutions of either substrate (FIG. 23). For ethanol this value corresponds to C/A, whereas for methanol this value corresponds to D/B. Plotting S2 as a function of S1 for various amounts of either substrate produces a straight line as can be seen from FIG. 23. A sample containing a mixture of ethanol and methanol provides a point located between those two lines (FIG. 24). A point located outside those lines indicates a sample interference or an experimental error.

It is also possible to graphically determine the respective concentrations of the substrates by drawing a line parallel to the line representing pure methanol over the point of the unknown sample (FIG. 24). The parallel line intercepts the line representing pure ethanol and generates two segments corresponding to the respective amounts of ethanol and methanol (FIG. 24).

Data Analysis by Third Degree Polynomials

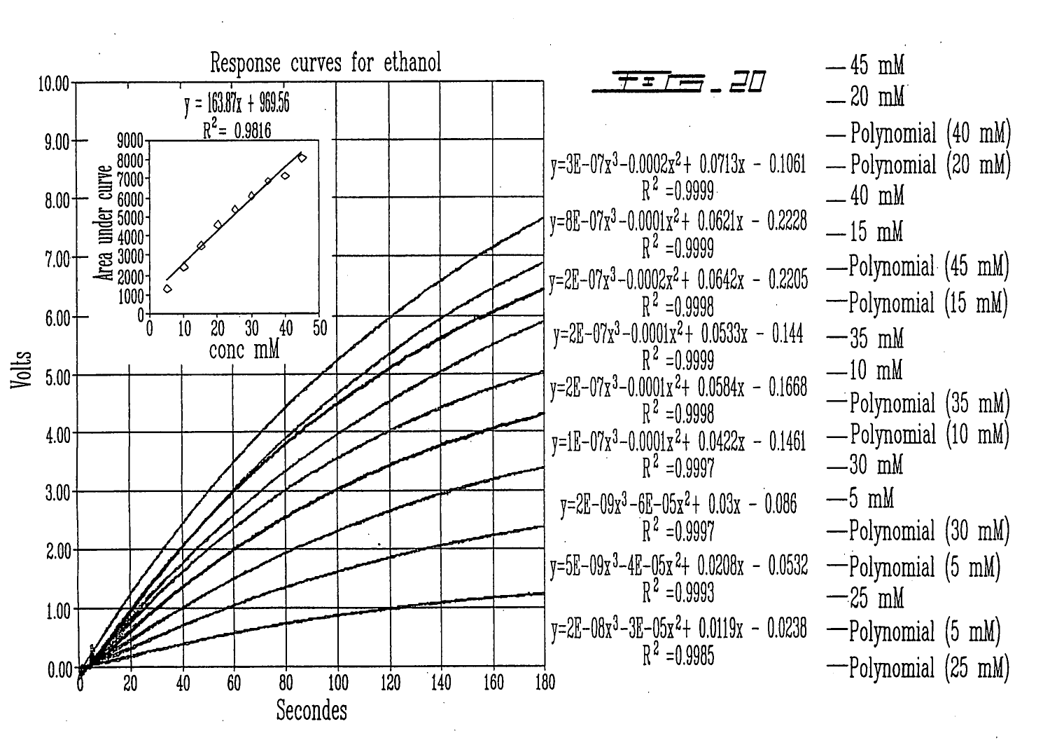

Regular fitting algorithms, like the one available in Microsoft Excel, can be used to fit a polynomial of the form y=a+bx+cx2+dx3 to the signal generated by the electrode as a function of time. Excellent correlation coefficients (better than 0.99) were experimentally obtained for methanol, ethanol and mixture thereof (FIGS. 19 and 20). It is thus possible to summarize multiple time data into a simple representative equation. A comparison with standard experimental curves can then be used to directly derive the amount of methanol and ethanol in any unknown sample.

When the enzyme is trapped near the electrode, the diffusion of peroxide towards the electrode is minimal, and it is therefore possible to obtain a reaction kinetics that is limited mainly by the enzyme. Furthermore, it is possible to achieve a first order reaction kinetics, with the current being directly proportional to the alcohol concentration. The enzyme kinetics are different for methanol and ethanol, and, as such, this property can be used to differentiate between the two alcohols. Since methanol reacts more rapidly than ethanol, its concentration over time decreases more rapidly.

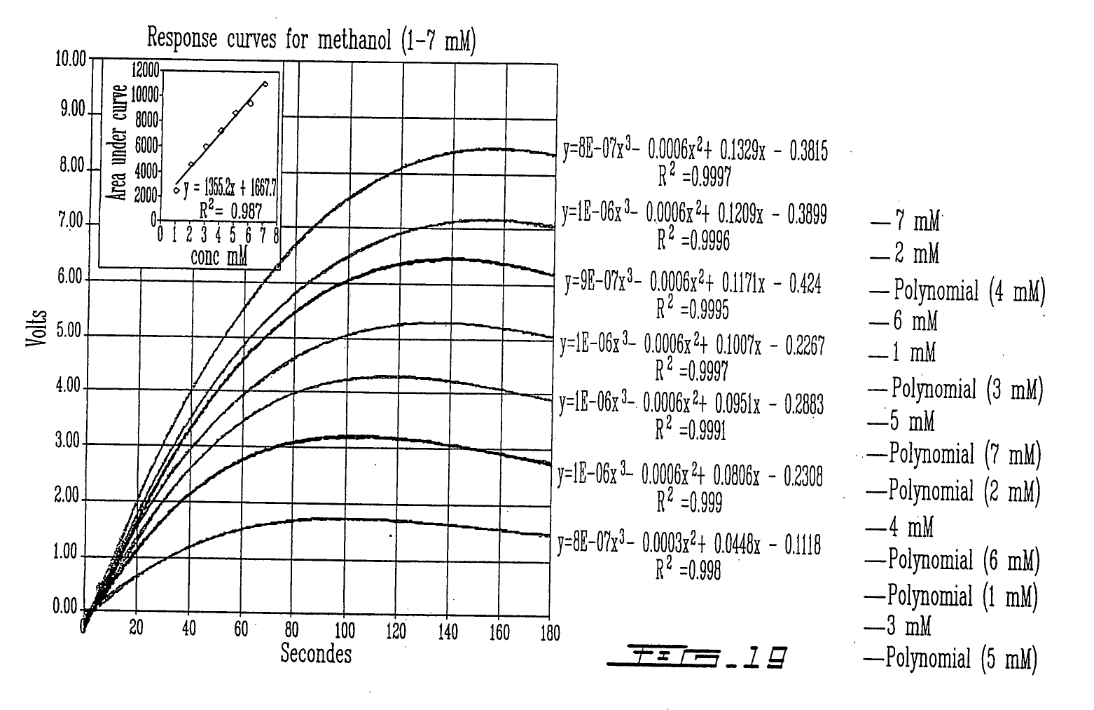

In a specific embodiment, the invention was conducted as follows: 10 μL of enzyme (Pichia pastoris; A-2404 Sigma) at 2 U/mL in a 400 mM potassium phosphate buffer (pH 7.2), were deposited onto the electrode, followed by 2 μL of a solution of alcohol; pure ethanol at various concentrations (4, 8, and 12 mM), pure methanol at various concentrations (4, 8, and 12 mM) and 1:1 mixtures of ethanol and methanol achieving final total concentrations of (4, 8, and 12 mM). The data, obtained as raw voltage measurements at the electrode are depicted in FIG. 18. The experiment was conducted using a Pt/Ag electrode strip, having no enzyme embedded.

In another specific embodiment of the present invention, in order to evaluate both the ethanol and methanol proportions, as well as their concentrations, the slope of the voltage curves at 4500 milliseconds is calculated. This value is then plotted against the absolute value at 4500 milliseconds, which allows for the estimation of the total alcohol concentration in an unknown solution, as well as for the determination of the relative proportions of both ethanol and methanol in the unknown solution (FIG. 18). The line connecting the diamond symbols, is illustrative of pure ethanol at various concentrations; the line connecting the squares is illustrative of pure methanol at various concentrations; the line connecting the triangles is illustrative of 50:50 mixtures of ethanol and methanol at various concentrations; and the dots represent various ethanol and methanol mixtures at the indicated concentrations. The total amount of each alcohol in a given mixture was determined using the dotted lines (the position with regards to each of the previously described lines provides for relative proportions of ethanol and methanol in a given mixture).

Tests aiming to improve the observed signals were carried out. Low concentrations of SDS (0.1%) allow for a uniform deposit of a sample on the surface of the working electrode, while simultaneously increasing the enzyme activity by a factor of 110%.

Although the present invention has been described hereinabove by way of preferred embodiments thereof, it can be modified, without departing from the spirit and nature of the subject invention as defined in the appended claims.

1. An analyzer for simultaneously detecting and measuring the concentration of two related analytes, said analytes being substrates for a common enzyme, comprising:
(a) an enzymatic reaction monitoring component including a support base, a mixed electrode system consisting of a working electrode, an auxiliary electrode and a reference electrode, said mixed electrode system being supported by said support base, and an enzymatic reaction means incorporating said enzyme, said enzymatic reaction means being disposed on said mixed electrode system; whereby, when said enzymatic reaction means is placed in contact with a liquid sample containing said two related analytes, said two related analytes chemically react with said enzyme to produce an electronic signal directly related to the concentration of each of said two related analytes in said liquid sample;
(b) a detector including a sensor, said detector being connected to said enzymatic reaction monitoring component and capable of continuously detecting and amplifying said electronic signal to produce amplified signals; and
(c) a data processor capable of converting the amplified signals into numerical data representative of the concentration of each of said two related analytes.

2. An analyzer as defined in claim 1, wherein said working electrode and said auxiliary electrode are composed of platinum, and wherein said reference electrode is composed of silver.

3. An analyzer as defined in claim 2, wherein said enzymatic reaction means comprises a layer of a permeable polymer on which is bound a layer including said enzyme, said layer being deposited on said mixed electrode system, and a protective membrane impregnable with a buffer solution and reagents capable of promoting said enzymatic reaction, said protective membrane being disposed over said layer of a permeable polymer.

4. An analyzer as defined in claim 3, wherein said permeable polymer is selected from the group consisting of polylysine, poly(4-styrene sulfonate), polyethylene glycol, perfluorosulfonic acid polymers and agarose.

5. An analyzer as defined in claim 4, wherein said reagents include electron transfer reagents selected from the group consisting of p-phenylenediamine, peroxidase and ferrocene derivatives.

6. An analyzer as defined in claim 5, wherein said ferrocene derivatives include ferrocene dicarboxylic acid, and ferrocene monocarboxylic acid.

7. An analyzer as defined in claim 6, wherein said buffer solution is selected from the group consisting of phosphates, saline phosphate buffers (phosphates+NaCl), TRIS-HCl, Hepes, with or without EDTA, and a wetting agent such as SDS, Triton X-100 and Tween 20.

8. An analyser as defined in claim 7, wherein said enzymatic reaction monitoring component is a disposable electrode.

9. An analyzer as defined in claim 2, wherein said enzymatic reaction means comprises a reagent well capable of receiving a buffer solution including said enzyme, said liquid sample, and optionally reagents capable of promoting said enzymatic reaction.

10. An analyzer as defined in claim 9, wherein said reagents include electron transfer reagents selected from the group consisting of p-phenylenediamine, peroxidase and ferrocene derivatives.

11. An analyzer as defined in claim 10, wherein said ferrocene derivatives include ferrocene dicarboxylic acid, and ferrocene monocarboxylic acid.

12. An analyzer as defined in claim 11, wherein said buffer solution is selected from the group consisting of phosphates, saline phosphate buffers (phosphates+NaCl), TRIS-HCl, Hepes, with or without EDTA, and a wetting agent such as SDS, Triton X-100 and Tween 20.

13. An analyser as defined in claim 12, wherein said enzymatic reaction monitoring component is a permanent electrode.

14. An analyzer as defined in claim 8, wherein said enzyme is an oxidase.

15. An analyser as defined in claim 14, wherein said oxidase is alcohol oxidase.

16. An analyzer as defined in claim 15, wherein said related analytes are methanol and ethanol.

17. An analyzer as defined in claim 16, wherein said liquid sample is a biological specimen selected from the group consisting of saliva, blood or serum.

18. An analyzer as defined in claim 17, wherein said support base is composed of any suitable material capable of supporting said mixed electrode system.

19. An analyzer as defined in claim 18, wherein said support base is composed of plastic.

20. An analyzer as defined in claim 8, wherein said analyzer is a portable analyzer.

21. An analyzer as defined in claim 13, wherein said analyzer is a non-portable analyzer.

22. An analyzer as defined in claim 20, for use in point-of-care units, in laboratories, in police services, in forensic applications and in industrial applications.

23. A method for simultaneously detecting and measuring the concentration of two related analytes in a sample, said related analytes being substrates for a common enzyme, wherein said enzyme reacts with said related analytes following specific different reaction kinetics, and wherein said method comprises:
(a) reacting a plurality of reference samples having known concentrations and proportions of said related analytes, said proportions ranging from 0 to 100% of a first analyte to 100% to 0% of another related analyte, with said enzyme;
(b) establishing a kinetic profile having at least two points for each of said plurality of reference samples; and
(c) reacting a test sample comprising an unknown concentration and proportion of said related analytes with said enzyme and determining the concentration of said related compounds in said test sample using said established kinetic profiles.

24. A method as defined in claim 23, wherein said unknown concentration of said related analytes is established using multiple regression analysis of said kinetic profile.

25. A method as defined in claim 23, wherein said unknown concentration of said related analytes is established using reaction kinetics equations.

26. A method as defined in claim 24, wherein said related analytes are methanol and ethanol.

27. An enzymatic reaction monitoring component for simultaneously detecting and measuring the concentration of two related analytes, said analytes being substrates for a common enzyme, comprising:
(a) a support base;
(b) a mixed electrode system consisting of a working electrode, an auxiliary electrode and a reference electrode, said mixed electrode system being supported by said support base; and
(c) an enzymatic reaction means incorporating said enzyme, said enzymatic reaction means being disposed on said mixed electrode system; whereby, when said enzymatic reaction means is placed in contact with a liquid sample containing said two related analytes, said two related analytes chemically react with said enzyme to produce an electronic signal directly related to the concentration of each of said two related analytes in said liquid sample.

28. An enzymatic reaction monitoring component as defined in claim 27, wherein said working electrode and said auxiliary electrode are composed of platinum, and wherein said reference electrode is composed of silver.

29. An enzymatic reaction monitoring component as defined in claim 28, wherein said enzymatic reaction means comprises a layer of a permeable polymer on which is bound an enzyme layer, said enzyme layer being deposited on said mixed electrode system, and a protective membrane impregnable with a buffer solution and reagents capable of promoting said enzymatic reaction, said protective membrane being disposed over said layer of a permeable polymer.

30. An enzymatic reaction monitoring component as defined in claim 29, wherein said permeable polymer is selected from the group consisting of polylysine, poly(4-styrene sulfonate), polyethylene glycol, perfluorosulfonic acid polymers and agarose.

31. An enzymatic reaction monitoring component as defined in claim 30, wherein said reagents include electron transfer reagents selected from the group consisting of p-phenylenediamine, peroxidase and ferrocene derivatives.

32. An enzymatic reaction monitoring component as defined in claim 31, wherein said ferrocene derivatives include ferrocene dicarboxylic acid, and ferrocene monocarboxylic acid.

33. An enzymatic reaction monitoring component as defined in claim 32, wherein said buffer solution is selected from the group consisting of phosphates, saline phosphate buffers (phosphates+NaCl), TRIS-HCl, Hepes, with or without EDTA, and a wetting agent such as SDS, Triton X-100 and Tween 20.

34. An enzymatic reaction monitoring component as defined in claim 33, wherein said enzymatic reaction monitoring component is a disposable electrode.

35. An enzymatic reaction monitoring component as defined in claim 28, wherein enzymatic reaction means comprises a reagent well capable of receiving a buffer solution including said enzyme, said liquid sample, and optionally reagents capable of promoting said enzymatic reaction.

36. An enzymatic reaction monitoring component as defined in claim 35, wherein said reagents include electron transfer reagents selected from the group consisting of p-phenylenediamine, peroxidase and ferrocene derivatives.

37. An enzymatic reaction monitoring component as defined in claim 36, wherein said ferrocene derivatives include ferrocene dicarboxylic acid, and ferrocene monocarboxylic acid.

38. An enzymatic reaction monitoring component as defined in claim 37, wherein said buffer solution is selected from the group consisting of phosphates, saline phosphate buffers (phosphates+NaCl), TRIS-HCl, Hepes, with or without EDTA, and a wetting agent such as SDS, Triton X-100 and Tween 20.

39. An enzymatic reaction monitoring component as defined in claim 38, wherein said enzymatic reaction monitoring component is a permanent electrode.

40. An enzymatic reaction monitoring component as defined in claim 34, wherein said enzyme is an oxidase.

41. An enzymatic reaction monitoring component as defined in claim 40, wherein said oxidase is alcohol oxidase.

42. An enzymatic reaction monitoring component as defined in claim 41, wherein said related analytes are methanol and ethanol.

43. An enzymatic reaction monitoring component as defined in claim 42, wherein said liquid sample is a biological specimen selected from the group consisting of saliva, blood or serum.

44. An enzymatic reaction monitoring component as defined in claim 43, wherein said support base is composed of any suitable material capable of supporting said mixed electrode system.

45. An enzymatic reaction monitoring component as defined in claim 44, wherein said support base is composed of plastic.
